(19) United States
(12) Patent Application Publication
(10) Pub. No.: US 2005/0079155 A1
(43) Pub. Date: Apr. 14, 2005

(54) Generating protein pro-drugs using reversible PPG linkages

(75) Inventor: Shannon Marshall, San Francisco, CA (US)

Correspondence Address: Robin M. Silva, Esq.;Dorsey & Whitney LLP, Intellectual Property Department, Four Embarcadero Center, Suite 3400, San Francisco, CA, 94111-4187, US

(73) Assignee: Xencor, Inc.

(21) Appl. No.: 10/811,492

(22) Filed: Mar. 19, 2004

Related U.S. Application Data
(60) Provisional application No. 60/456,094, filed on Mar. 20, 2003.

(51) Int. Cl. A61K 39/00; A61K 38/21

(52) U.S. Cl. 424/85.2; 424/192.1; 424/85.6

(57) ABSTRACT

The invention relates to methods for modulating the side effects, including immunogenicity, of a protein therapeutic via derivatization with a protein protecting group using reversible or labile linkages.

This application claims benefit under 35 U.S.C. §119(e) to U.S. Ser. No. 60/456,094, filed Mar. 20, 2003, hereby incorporated by reference in its entirety.

### FIELD OF THE INVENTION

The invention relates to methods for modulating the immunogenicity and side effects of protein therapeutics by derivatization using reversible or labile linkages. The invention further relates to the use of protein therapeutics having reduced immunogenicity and side effects as well as treatment with the same.

### BACKGROUND OF THE INVENTION

Many protein therapeutics induce unwanted side effects. In a number of cases, these side effects result from receptor binding or protein activity at or close to the site of administration. For example, immunomodulatory protein therapeutics may stimulate the immune system, leading to unwanted immunogenicity of the therapeutic. Binding to receptors on the surface of antigen presenting cells may also lead to uptake, processing, and presentation of peptides derived from a protein therapeutic; such steps may lead to T-cell activation, B-cell activation, and antibody production. Furthermore, protein therapeutics that act as growth factors may induce unwanted proliferation or differentiation of cells at or close to the site of administration.

Accordingly, methods to limit the activity of a protein therapeutic at or close to the site of administration, including but not limited to its ability to bind receptors, may be used to reduce unwanted side effects of protein therapeutics.

Modification of protein therapeutics with polyethylene glycol (PEGylation) is commonly pursued to improve the pharmacokinetics of protein therapeutics; PEGylation may also reduce immunogenicity. One apparent disadvantage of many PEGylated protein therapeutics is that they have significantly reduced specific activity relative to the unmodified proteins (Hershfield et. al. Proc. Nat. Acad. Sci. USA 88:7185-7189; Bailon. et al. 2001 Bioconjug. Chem. 12, 195-202; He et al. 1999 Life Sci. 65:355-368; Wang et al. Adv. Drug Deliv. Rev. 2002 54:547-570). While the activity loss associated with PEGylation has traditionally been considered a problem to be overcome, it may actually prove beneficial in reducing side effects that are caused by unwanted activity of the protein therapeutic at or close to the site of administration.

Accordingly it is an object of the present invention to provide protein therapeutics with reduced side effects, as well as methods of making and using the same.

### SUMMARY OF THE INVENTION

In accordance with the object outlined above, the present invention is directed to a method to reduce or block the activity of a protein therapeutic, including but not limited to its receptor binding activity, by reversibly attaching one or more protein protecting groups (PPGs). Such modification may decrease side effects including but not limited to allergenicity, hypersensitivity responses, production of anti-drug antibodies, and unwanted cell proliferation or differentiation. To preserve the desired therapeutic activity of the protein, the PPGs are attached using reversible or labile covalent linkages. The linkage chemistry is selected so that the protein is substantially inactive when administered, and substantially activated following absorption from the site of administration but prior to excretion. This invention further comprises protein therapeutics comprising one or more PPGs, treatments in which such a modified protein therapeutic is administered to a patient, and to methods of making and using the same.

### BRIEF DESCRIPTION OF THE DRAWINGS

FIG. 1: Shows the sequence of human TPO cytokine domain. Especially preferred sites for PPG attachment are indicated in bold. (SEQ ID NO: 1)

FIG. 2: Shows the sequence of human BMP-7. Especially preferred sites for PPG attachment are indicated in bold. (SEQ ID NO:2)

FIG. 3: Shows the sequence of human interferon beta. Especially preferred sites for PPG attachment are indicated in bold. (SEQ ID NO:2)

FIG. 4: Shows the sequence of human CNTF. Especially preferred sites for PPG attachment are indicated in bold. (SEQ ID NO:2)

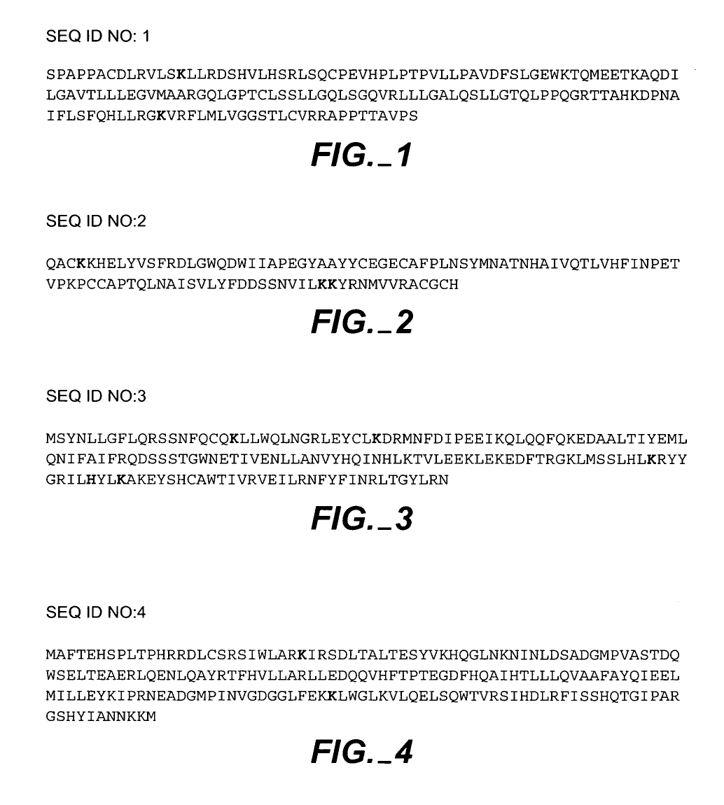

### DETAILED DESCRIPTION OF THE INVENTION

By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

By “pharmaceutically acceptable carrier” is meant agents (e.g. exipients) that facilitate the formulation and delivery of the compositions of the inventions. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP), and include acid and base salts. By “pharmaceutically acceptable acid addition salt” as used herein refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. By “pharmaceutically acceptable base addition salts” as used herein is meant to include those salts derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

By “protein” herein is meant a molecule comprising at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)]. For example, homo-phenylalanine, citrulline, and noreleucine are considered amino acids for the purposes of the invention. “Amino acid” also includes amino acid residues such as proline and hydroxyproline. Both D- and L-amino acids may be utilized.

By “protein protecting group” or “PPG” and grammatical equivalents as used herein means any chemical entity that can be reversibly covalently linked to a protein and substantially interfere with the binding of that protein to one or more receptors on the surface of Antigen Presenting Cells (APCs) or other cells present at or close to the site of administration.

By “reduced immunogenicity” and grammatical equivalents herein is meant a decreased ability to activate the immune system, when compared to the wild type protein. For example, a PPG-conjugated protein may be said to have “reduced immunogenicity” if it elicits neutralizing or non-neutralizing antibodies in lower titer or in fewer patients than the unmodified protein. In a preferred embodiment, the probability of raising neutralizing antibodies is decreased by at least 5%, with at least 50% or 90% decreases being especially preferred. So, if an unmodified protein produces an immune response in 10% of patients, a PPG-conjugated version of that protein with reduced immunogenicity would produce an immune response in not more than 9.5% of patients, with less than about 5% or less than about 1% being especially preferred. A PPG-conjugated protein also may be said to have “reduced immunogenicity” if it shows decreased binding to one or more MHC alleles or if it induces T-cell activation in a decreased fraction of patients relative to the unmodified protein. In a preferred embodiment, the probability of T-cell activation is decreased by at least about 5%, with at least about 50% to about 90% decreases being especially preferred.

Accordingly, the present invention provides compositions comprising a prodrug agent comprising proteins covalently linked via a labile linker to a substantially non-immunogenic polymer. “Prodrug” in this context means a therapeutic protein that has a covalently attached polymer as outlined herein that results in the a decrease in the bioactivity of the protein, but is activated upon cleavage of the polymer off the protein, restoring activity.

Identification of Proteins Suited for PPG Modification

Protein therapeutics that are suited for PPG modification include any protein therapeutic whose activity, at or close to the site of administration, is associated with undesired side effects. In many cases, the undesired side effect is unwanted immunogenicity, although other side effects are also possible, including other types of inflammation (swelling, itching, etc.), as well as loss of bioactivity upon administration (e.g. protease cleavage at the site of administration that deactives the drug). As will be appreciated by those in the art, there are a number of therapeutic proteins which are either known to or capable of producing undesired side effects at or close to the site of administration, e.g. the site of subcutaneous injection, inhalation, etc.

In a preferred embodiment, the protein modified by addition of one or more PPGs is capable of activating or stimulating antigen presenting cells or binding receptors present on the surface of antigen presenting cells, including but not limited to dendritic cells, macrophages, and B-cells. Proteins that are capable of activating or stimulating antigen presenting cells or binding receptors present on the surface of antigen presenting cells include, but are not limited to, antibodies, ciliary neurotrophic factor, endothelin, interleukin-1, interleukin-4, interleukin-8, interleukin-13, interferon-gamma, macrophage-inflammatory protein, macrophage stimulating protein, matrix metalloproteinases, thrombopoietin, transforming growth factor-beta, tumor necrosis factor-alpha, and tumor necrosis factor-beta. These include both wild-type proteins and derivatives thereof.

In another preferred embodiment, the protein modified by addition of one or more PPGs is capable of activating or stimulating T-cells. Such activation may lead to delayed-type hypersensitivity response or to B-cell activation and antibody production. Proteins that are capable of activating or stimulating T-cells include, but are not limited to, interferon-gamma, interleukin-1, interleukin-2, interleukin-4, interleukin-6, interleukin-7, interleukin-9, interleukin-12, and interleukin-23. These include both wild-type proteins and derivatives thereof.

Any other protein with immunostimulatory activity may also benefit from PPG attachment. Additional proteins with immunostimulatory activity include, but are not limited to, antibodies, complement pathway proteins, interferon alpha, interferon beta, interferon gamma, interleukin-2, interleukin-4, interleukin-5, interleukin-11, interleukin-12, interleukin-23, GM-CSF, additional chemokines, and the like. These include both wild-type proteins and derivatives thereof.

In an additional preferred embodiment, the protein modified by addition of one or more PPGs is a growth factor that is capable of inducing unwanted proliferation or differentiation of cells located at or close to the injection site. For example, bone morphogenic protein-7 is capable of inducing ectopic bone formation at subcutaneous injection sites. Protein growth factors include, but are not limited to, vascular endothelial growth factor, tumor growth factor-beta, insulin-like growth factor, fibroblast growth factors (e.g. FGF-2, FGF-7, and FGF-10), and bone morphogenic proteins (e.g. BMP-2, BMP-4, BMP-6, and BMP-7). These include both wild-type proteins and derivatives thereof.

Derivatization with Protein Protecting Groups

Preferred Properties of the PPG

In a preferred embodiment, the PPG described herein will possess the following characteristics: water-solubility, substantially non-immunogenic in the patient, a lack of ability to significantly bind or activate dendritic cells, and a lack of -toxicity (e.g. physiologically acceptable). By “substantially non-immunogenic” herein is meant that the PPG comprises little or no immunogenicity upon administration to a patient.

Suitable PPGs may be polymers, including but not limited to polyalkane glycols, dextrans, polysaccharides, and peptides, as well as non-polymers including but not limited to ethylene groups, alkanes, lipids, sugars, and amino acids.

A preferred PPG is PEG (polyethylene glycol). Polyethylene glycol (PEG) is a highly flexible and soluble polymer that has gained widespread acceptance as a chemical modification for therapeutic proteins. PEG attachment (PEGylation) to a protein can increase serum half-life by conferring a dramatic increase in effective molecular size. PEGylation can also reduce immunogenicity by blocking access to antibody epitopes, as well as decrease aggregation and minimize fluctuations in serum concentration (Roberts et al. Adv. Drug Deliv. Rev. 2002 54:459-476; Kinstler et al. Adv. Drug Deliv. Rev. 2002 54). Several PEGylated protein therapeutics are currently on the market or in late-stage clinical trials, including one that utilizes reversible linkages, Schering-Plough's PEG-Intron® (peginterferon alpha-2b).

Any of a variety of pharmaceutically or physiologically acceptable PEG polymers may be used in the present invention. PEG polymers of any size and branching structure may be used, so long as they substantially reduce the activity of the protein upon attachment.

Preferred Linkages between the Protein and the Protein Protecting Group

The linkage between the PPG described herein and the therapeutic protein must be covalent and reversible. That is, under physiological conditions, the linkage between the PPG and the protein must be labile, e.g. cleavable. Using labile linkages for PPG attachment allows regeneration of active therapeutic protein over time, preferably following absorption from the site of administration. Proper tuning of the reactivity of the linkage [for example Greenwald et. al. Bioconjug. Chem. 2003 14:395-403] will enable the generation of a modified therapeutic protein that is substantially incapable of receptor binding immediately following administration but substantially active for a sufficient time prior to elimination to confer desired therapeutic efficacy. In preferred embodiments, the reversible linkage is such that upon release of the PPG, the drug is regenerated, e.g. there are no non-drug atoms attached to the protein.

PEG may be attached to proteins using a variety of chemistries. In general, the proteins are attached to the PPG through the use of functional groups on each that can then be used for attachment. Preferred functional groups for attachment are amino groups, carboxy groups, oxo groups and thiol groups. The functional group on the PPG and the functional group on the protein can then be attached, either directly or indirectly through the use of an additional linker. Linkers are well known in the art; for example, homo-or hetero-bifunctional linkers as are well known (see 1994 Pierce Chemical Company catalog, technical section on cross-linkers, pages 155-200, incorporated herein by reference). Preferred linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C2 alkene being especially preferred. The linker may also be a sulfone group, forming sulfonamide linkages.

Preferred embodiments utilizes linkages that are formed between the polymer (e.g. PEG) and primary amines (lysine or arginine side chains or the protein N-terminus), thiols (cysteine residues), or histidines. It is also possible to PEGylate carboxylate and hydroxyl groups. Lysine occurs frequently on the surface of proteins, so PEGylation of lysine side chains generally produces a mix of PEGylation products. Since the pKₐ of the N-terminus is significantly different than the pKₐ of a typical lysine side chain, it is possible to specifically target the N-terminus for modification, or, conversely, avoid an N-terminal attachment. Similarly, as most proteins contain very few free cysteine residues, cysteines (naturally occurring or engineered) are commonly targeted for site-specific PEGylation.

In a preferred embodiment, reversible attachment methods are used that allow PPG to be released from the protein without any non-protein atoms remaining attached to the protein. Examples of chemistries that allow for complete PPG release include, but are not limited to, PEG maleic anhydride and mPEG benzamide succinamidyl carbamates for amines and PEG orthopyridyl disulfide for cysteines (see for example Roberts et al. Adv. Drug Deliv. Rev. 2002 54:459-476; Lee et. al. Bioconjug. Chem. 2001 12:163-169; Veronese, Biomaterials 2001 22: 405-417, Greenwald et. al., J. Bioconjug. Chem. 2003 14:395-403; Roberts and Harris, J. Pharm. Sci. 1998 87:1440-1445).

In a preferred embodiment, the attachment is a cleavage site, comprising an enzyme substrate moiety. Suitable classes of enzymes include, but are not limited to, hydrolases such as proteases, carbohydrases, lipases and nucleases, and in some cases isomerases such as racemases, epimerases, tautomerases, or mutases; transferases, kinases and phophatases. Particularly preferred are protease cleavage sites, which allow for the release of the PPG upon exposure to an enzyme. Suitable enzymes will depend on the site of administration or required activation site. Enzymes such as lactase, maltase, sucrase or invertase, cellulase, a-amylase, aldolases, glycogen phosphorylase, kinases such as hexokinase, proteases such as serine, cysteine, aspartyl and metalloproteases may also be used to cleave the PPG, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, such as interleukin-converting enzyme (ICE). Similarly, the cleavage sites for matrix metalloproteinases (MMPs) can used. In some cases, for example in the treatment of bacterial and viral infections, cleavage sites characteristic to bacterial and viral enzymes can be used. A variety of known protease cleavage sites are well known in the art.

Preferred Attachment Sites

As the objective of the present invention is limiting unwanted protein activity at or near the injection site, any site may be used for PPG attachment so long as the resulting protein-PPG construct exhibits substantially decreased activity relative to the unmodified protein. It is possible to attach the PPG to a single site or to multiple sites. Furthermore, some methods will produce a mixture of products in which the location or number of PPG attachment sites is heterogeneous.

In a preferred embodiment, one or more cysteine, histidine, or lysine residues comprises a PPG attachment site. The N-terminus may also comprise a PPG attachment site. The cysteine, histidine, or lysine residues may be present in the parent protein sequence. It is also possible to modify the parent protein sequence by incorporating one or more PPG attachment sites.

In a preferred embodiment, PPG attachment sites are rationally selected to maximally occlude one or more binding or catalytic sites that are involved in protein function. For example, critical residues may be identified using mutagenesis approaches. It is also possible to model the protein and PPG to determine the effects of PPG attachment (see U.S. Ser. No. 60/459,094 filed Mar. 31, 2003 and U.S. Ser. No. ______, filed Mar. 31, 2004, entitled METHODS FOR RATIONAL PEGYLATION OF PROTEINS, both hereby incorporated by reference in their entirety.)

In a preferred embodiment, the PPG attachment site is chosen to be at an internal residue, rather than the N- or C-terminal amino acids, although in some cases either or both of the N- or C-terminal amino acids can be used. Preferably, the PPG attachment site is a surface residue, as defined within WO 01/59066, hereby incorporated by reference in its entirety, and specifically the discussion of surface residues. In preferred embodiments, the surface residue is at or near the active site such that effective blocking of the active site (whether an enzymatic active site, a binding site in the case of a receptor or ligand, etc.) can occur. The elucidation of a suitable site will be done as is known in the art, for example using the three dimensional structure, if known for the protein of interest or a homolog, or using computation means such as outlined in the above identified WO 01/59066.

Human TPO contains multiple lysine residues that have been implicated in binding to the mpl ligand. These residues comprise preferred sites for PPG attachment. The most preferred positions for PPG attachment include, but are not limited to, K14 and K137.

Human BMP-7 comprises several reactive residues that are located at the binding site for the type I or type II receptor. These residues comprise preferred sites for PPG attachment. The most preferred positions for PPG attachment include, but are not limited to, K39, K126, and K127.

Human IFN-β comprises several reactive residues that have been implicated in receptor binding [Runkel et. al. Biochem. 2000 39: 2538-2551]. These residues comprise preferred sites for PPG attachment. The most preferred positions for PPG attachment include, but are not limited to, K19, K33, K123, H131, and K134.

Human CNTF comprises several reactive residues that are located at the binding sites for its receptors [DiMarco et. al. Proc. Nat. Acad. Sci. 1996 93: 9247-9252; Panayotatos et. al. J. Biol. Chem. 1995 270:14007-14014]. These residues comprise preferred sites for PPG attachment. The most preferred positions for PPG attachment include, but are not limited to, K26 and K155.

PPG Modification of TPO

In an especially preferred embodiment, TPO (thrombopoietin, also called MGDF or mpl ligand) is modified by PPG addition. TPO is a cytokine that acts to promote platelet formation as well as the growth and differentiation of several myeloid lineages. TPO shows promise for the treatment of thrombocytopenia (low platelet count) resulting from a variety of causes. However, development of TPO has been hindered by its immunogenicity; unwanted immune responses have been observed following subcutaneous injection (Yang et. al. Blood 2002 98: 3241-3248). TPO variants that have been de-immunized by removing T-cell epitopes have been disclosed previously. (See, for example, U.S. Ser. Nos. 09/903,378; 10/039,170; 60/432,909; 10/339,788; 60/402,344; 60/406,232; and PCT/01/21823 and PCT/02/00165, all are hereby expressly incorporated by reference.) Here, a complementary approach to de-immunization is disclosed.

The TPO receptor (c-mpl) is present in dendritic cells (a type of APC), as well as in megakaryocytes and other myeloid cell types. Subcutaneous injection of TPO strongly activates the dendritic cells that are present at the injection site, causing inflammation and promoting TPO immunogenicity. Furthermore, receptor mediated endocytosis mediated by the c-mpl receptor may dramatically increase the amount of TPO that is consumed and presented by the APCs. In effect, TPO may behave as an adjuvant, thereby promoting its immunogenicity.

PPGs may be used to prevent TPO from binding its receptor when it is first injected, while maintaining desired TPO activity following absorption into the bloodstream. A TPO molecule appropriately derivatized with a PPG could constitute an active, non-immunogenic variant for stimulation of platelet formation and/or differentiation and development of additional myeloid lineages.

TPO has been modified previously by stable PEGylation of the N-terminus (See e.g., U.S. Pat. No. 5,795,569 and U.S. Pat. No. 5,766,581); interestingly this is the molecule that produced the unwanted immune responses discussed above. N-terminal PEGylation confers improved serum half-life and minimally reduces activity. To reduce immunogenicity, reactive groups located at or near the mpl receptor binding site may be targeted for reversible PEGylation. Such residues may be identified using any of a number of methods, including mutagenesis and analysis of the structure of TPO and related cytokines.

PPG Modification of BMP-7

In another especially preferred embodiment, BMP-7 (bone morphogenic protein 7, also called osteogenic protein-1 or OP-1) is modified by PPG attachment. BMP-7 is a growth factor that was initially identified for its role in bone development but also plays a role in regulating the differentiation, proliferation, chemotaxis, and apoptosis of a wide range of cell types [Balemans Dev. Biol. 2002 250: 231-250]. BMP-7 is used clinically to promote bone repair and healing; it is also in development for the treatment of renal fibrosis.

BMP-7 has been reported to elicit antibodies in 13-38% of patients [Koren, Curr. Parmaceut. Biotechnol. 2002 3: 349-360]. Furthermore, it has been observed to induce ectopic bone formation at subcutaneous and intramuscular injection sites in rodent studies[see Urist, Science 1965 150:893-899 and van de Putte Clin. Orthapaed. Rel. Res. 1965 275-270]. It would be desirable to generate a less immunogenic variant of BMP-7. Also, preventing unwanted ectopic bone formation could enable administration via subcutaneous or intramuscular injection.

BMP-7 binds to a number of type I and type II serine/threonine kinase receptors. Residues that mediate receptor interactions may be identified by analysis of the crystal structures of BMP-7 bound to ActRII [Greenwald Mol. Cell 2003 11: 605-617] and the crystal structure of BMP-2 bound to ALK-3 [Kirsch et. al. Nat. Struct. Biol. 2000 7: 492-496].

PPG Modification of IFN-β

In an additional especially preferred embodiment, IFN-β (interferon beta) is modified by PPG attachment. IFN-β has antiviral, antineoplastic, and immunomodulatory activities; it is used clinically to treat multiple sclerosis and has been investigated for a number of other therapeutic areas.

IFN-β has been reported to elicit neutralizing antibodies in a substantial portion of patients. Furthermore, it can induce injection-site reactions, some of which are likely due to delayed-type hypersensitivity responses. Accordingly, a less immunogenic variant of IFN-β may serve as a superior therapeutic agent.

IFN-β binds to the type I interferon receptor, which comprises two chains. The receptor is expressed by a wide variety of cell types, including many cell types that are present at most common sites of administration. Following receptor binding, IFN-β induces the expression of a number of interferon responsive gene products, including several pro-inflammatory cytokines. Substantially blocking this signal transduction cascade may promote a less inflammatory milieu at the site of administration, which would be expected to decrease the probability of unwanted immune responses.

PPG Modification of CNTF

In a further especially preferred embodiment, CNTF (ciliary neurotrophic factor, or Axokine® (Regeneron), a modified variant) is modified by PPG attachment. CNTF is under investigation as a treatment for obesity [Ettinger et. al. JAMA 2003 289:1826-1832].

During clinical trials, Axokine was observed to elicit neutralizing antibodies in the majority of patients tested, potentially limiting its safety or efficacy. Accordingly, a less immunogenic variant of CNTF or Axokine is desired.

CNTF binds to a number of receptors, including CNTFR, gp130, and LIFR [Gearing et. al. Proc. Nat. Acad. Sci. USA 1994 91:1119-1123]. Some or all of these receptors may be present on the surface of antigen presenting cells; for example gpl 30 and LIF are known to be expressed by APCs. Accordingly, binding to these receptors may promote uptake and hence immunogenicity of Axokine and CNTF.

Further Modifications of the Selected Protein

Therapeutic proteins that will be modified by the reversible addition of one or more PPGs may be subjected to additional modifications, including but not limited to those described below.

In a preferred embodiment, additional properties are optimized, including but not limited to MHC-binding affinity, receptor binding affinity, solubility, and stability. Furthermore, the amino acid sequence may be modified by adding or removing one or more residues which could serve as PPG attachment sites. It is also possible to truncate or circularly permute the protein to alter the location of the N-terminus, another possible PPG attachment site. The protein may also be modified by adding a fusion partner or tag.

In a most preferred embodiment, Protein Design Automation® (PDA™) technology is used to perform such further optimization (See U.S. Pat. Nos. 6,188,965; 6,269,312; 6,403,312; WO98/47089 and U.S. Ser. Nos. 60/104,612, 60/158,700, 60/181,630, 60/186,904, 09/419,351, 09/782,004 and 09/927,790, 60/347,772, and 10/218,102; 60/345,805; 60/373,453; 60/374,035; and WO02/25588 all references expressly incorporated herein in their entirety.)

In a further preferred embodiment, the protein is subjected to additional posttranslational modifications, including but not limited to stable PEGylation, glycosylation, lipidation, and synthetic modifications.

Protein Expression and Purification

The proteins that are to be modified by PPG attachment and nucleic acids encoding them may be produced by a number of methods known in the art.

In a preferred embodiment, nucleic acid encoding the desired protein is cloned into an appropriate expression vector and expressed in E. coli (see McDonald, J. R., Ko, C., Mismer, D., Smith, D. J. and Collins, F. Biochim. Biophys. Acta 1090: 70-80 (1991)). In an alternate preferred embodiment, the protein is expressed in mammalian cells, yeast, baculovirus, or in vitro expression systems. A number of expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed.

In a preferred embodiment, the protein is purified or isolated after expression. Standard purification methods include electrophoretic, molecular, immunological and chromatographic techniques, including ion exchange, hydrophobic, affinity, and reverse-phase HPLC chromatography, and chromatofocusing. For example, a protein variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed. (1994).

Characterization of the PPG-Protein Conjugate

In a preferred embodiment, following synthesis the PPG-protein conjugate is characterized for properties including but not limited to activity, stability, and pharmacokinetics.

The activity of the PPG-protein conjugate may be determined using any assay that is appropriate for the protein of interest, including but not limited to those assays disclosed below. In all cases, in a preferred embodiment activity is determined for three species: (1) unreacted protein, (2) protein with PPG attached, and (3) protein following PPG release. Release of a protein from PPG may be obtained by exposing the PPG-protein conjugate to conditions that favor breaking of the protein-PPG linkage; for example acidic conditions or reducing conditions.

Activity Assays for TPO

In a preferred embodiment, TPO proteins are analyzed for their ability to induce luciferase expression in an engineered TPO-responsive cell line, BAF-3 (Duffy et. al., J. Med. Chem. 2001 44:3730-3745). Briefly, the cells are transfected with genes encoding the TPO receptor and a luciferase reporter construct. The cells are treated with varying concentrations of wild type or variant TPO, and luminescence is measured.

In another preferred embodiment, TPO proteins are analyzed for their ability to sustain viability and growth of the TPO-responsive cell line M-07e (Brizzi et. al., Br. J. Haematol., 1990 76: 203-209). When stimulated with TPO, the growth of this megakaryocytoma-derived cell line, which constitutively expresses c-Mpl (the TPO-receptor) and other megakaryocyte markers, can be sustained in a concentration-dependent and saturable manner. A reliable, non-radioactive indicator for cell growth is Alamar Blue, a water-soluble non-toxic fluorometric/colorimetric proliferation indicator that measures cell metabolism (Shahan et. al., J. Immunol. Meth., 1994 175: 181-187). Cellular growth and metabolism reduces Alamar Blue, resulting in a blue-to-red color change. Non-viable or quiescent cells do not reduce Alamar Blue and thus no color change is observed.

Activity Assays for Interferon Beta

In a preferred embodiment, the activity of interferon beta proteins is analyzed using assays including but are not limited to reporter gene assays, receptor binding assays, antiviral activity assays, cytopathic effect inhibition assays, antiproliferative assays, immunomodulatory assays, and assays that monitor the induction of MHC molecules, all described in Meager, J. Immunol. Meth., 261:21-36 (2002).

Activity Assays for BMP-7

In a preferred embodiment, the activity of BMP-7 proteins is analyzed using receptor binding assays such as ELISA-based binding assays, Biacore® assays, or AlphaScreen™ assays. Receptors for BMP-7 proteins include ALK-2, ALK-3, ALK-6, ActRII, ActRIIb, and BMPRII.

In an alternate preferred embodiment, the activity of BMP-7 proteins is analyzed using reporter gene assays [for example Piek et. al. J. Cell. Physiol. 1999 180: 141-149].

In an additional preferred embodiment, the activity of BMP-7 proteins is analyzed using cellular differentiation assays. For example, treatment with BMP-7 induces chondroblastic, osteoblastic, and adipocytic differentiation in cell lines including but not limited to C1C12, TE-85, ATDC5, MC3T3-E1, and C₃H10T1/2 [Asahina et. al. Exp. Cell Res. 1996 222: 38-47; Cheng et. al. J. Bone Joint Surgery 2003 85: 1544-1552]. Similar assays may also be performed using tissues or animal models.

PPG Release Studies

In a preferred embodiment, the stability of the PPG-protein conjugate is determined in the formulation buffer and in serum. If necessary, the formulation conditions may be altered to ensure that the protein-PPG linkage is substantially intact prior to administration. Furthermore, if necessary the linkage chemistry may be changed to ensure that the kinetics of protein release in serum is sufficiently rapid to allow therapeutic efficacy.

A number of methods may be used to differentiate PPG-protein conjugates from unreacted or released protein. For example, methods that distinguish these species based on size or molecular weight may be used. In one embodiment, capillary electrophoresis is used [Roberts & Harris, J. Pharm. Sci. 1998 87: 1440-1445]. In an alternate embodiment, mass spectrometry methods are used. It is also possible to use non-denaturing gel electrophoresis. Alternatively, if the protein-PPG conjugages are inactive and the free proteins are active, methods that distinguish these species based on activity may be used. Another possibility is to quantitate with an antibody that is capable of binding to the free protein but substantially incapable of binding to the PPG-protein conjugate.

Pharmacokinetic Studies

In a preferred embodiment, the pharmacokinetic properties of the unreacted protein versus one or more PPG-modified proteins are characterized. Studies may be conducted in one or more animals including but not limited to mice, rats, primates, and humans, testing routes of administration including but not limited to intravenous injection, subcutaneous injection, intramuscular injection, and inhalation. As is known in the art, the serum concentration of protein is determined at a number of time points in order to determine values for pharmacokinetic parameters including but not limited to serum half-life, maximum serum concentration, and bioavailability. In a preferred embodiment, the serum concentration of PPG-attached and released protein is determined at each timepoint. In a preferred embodiment, additional biomarkers are also assayed; for example for IFNβ the expression of IFN-inducible proteins may be monitored as a function of time.

Administration and Treatment using PPG-Conjugated Proteins

Once made, the PPG-conjugated proteins of the invention find use in a number of applications. In a preferred embodiment, a PPG-conjugated protein is administered to a patient to treat a disorder that is responsive to that protein.

The administration of the PPG-conjugated protein of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. Depending upon the manner of introduction, the pharmaceutical composition may be formulated in a variety of ways.

The pharmaceutical compositions of the present invention comprise a PPG-conjugated protein in a form suitable for administration to a patient. In the preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

Combinations of pharmaceutical compositions may be administered. Moreover, the compositions may be administered in combination with other therapeutics.

All references cited herein are hereby expressly incorporated by reference in their entirety.

While the present invention has been described in terms of preferred embodiments, it is understood that variation and modifications will occur to those skilled in the art. Therefore, it is intended that the appended claims cover all such equivalent variations and modifications, which come within the scope of the invention as claimed.

### EXAMPLES

### Example 1

### Reversible PEGylation of Interferon-Beta

Production of Interferon Beta in E. coli

Sequence verified clones in pET28a were transformed into BL21(DE3) star cells (commercially available from Invitrogen) and cultures were grown in auto-inducing media, a rich medium for growth with little or no induction during log phase and auto-induction of expression as the culture approaches saturation. Media components include 25 mM (NH₄)₂SO₄, 50 mM KH₂PO₄, 50 mM Na₂HPO₄, 1 mM MgSO₄, 0.5% glycerol, 0.05% glucose, 0.2% alpha-lactose, 0.1% tryptone, and 0.05% yeast extract. The cultures were grown for 7 hours to an OD between 4 and 5 and cells harvested by centrifugation. Cells were lysed by sonication, inclusion pellets denatured in 8M guanidine HCl and bound to a column containing Ni-NTA resin. A dilution series of guanidine HCl with decreasing pH was used to purify and refold the protein.

An alternative method for purification of clones with and without the N-terminal 6-His tag was followed as disclosed in U.S. Pat. No. 4,462,940, Lin et al, Meth. Enzymol. 119:183-192.

Conjugation Reaction

To conjugate PEG to lysine residues, the protein and activated PEG will be allowed to react at basic pH (7.5-10.0) for 30 minutes —4 hours at a temperature between 4° C. and 37° C. PEG-maleic anhydride and PEG-benzamide succinamidyl carbamate chemistries are tested, and PEG-5000 will be used. The molar ratio of PEG to protein will be determined by the number of PEG groups whose addition is desired; appropriate ranges are from 1:1 to 50:1. Reaction progress may be monitored to determine the optimal reaction time. After the reaction has progressed sufficiently, it will be quenched by reducing the pH of the buffer. The products may be purified by dialysis.

### Example 2

### Characterization of the Protein-PPG Conjugate

Generation of de-PEGylated Material

PEGylated interferon beta will be de-PEGylated by reducing the buffer pH to 5.0 and stirring for one hour.

Activity Assays

A standard ISRE (interferon-stimulated response element) reporter assay will be used to determine the activity of unreacted interferon beta, PEGylated interferon beta, and de-PEGylated interferon beta. In this assay, 293T cells which constitutively express the type I interferon receptor will be transiently transfected with an ISRE-luciferase vector (pISRE-luc, commercially available from Clontech). Twelve hours after transfection, the cells will be treated with a dilution series of concentrations for each interferon beta species. Proteins which bind the interferon receptor and trigger the JAK/STAT signal transduction cascade activate transcription of the luciferase gene operably linked to the ISRE. Luciferase activity will be detected using the Steady-Glo® Luciferase Assay System (commercially available from Promega) with the TopCount NXT™ microplate reader used to measure luminescence.

Standard antiviral assays will also be used to determine the activity of unreacted interferon beta, PEGylated interferon beta, and de-PEGylated interferon beta. The antiviral assay will be performed using A549 human lung carcinoma cells (ATCC CCL185). The cells will be added to 96-well plates at a density of 3×10⁵ cells/mL and a volume of 100 μL. After 24 hours, the cells will be treated with interferon beta concentrations ranging from 1 pg/mL to 1 ng/mL; each condition will be tested in triplicate. After 24 hours, the cells will be treated with encephalomyocerditis virus. After 48 hours, the cells will be treated with thiazolyl blue to determine the number of viable cells.

Stability Assays

Stability of protein-PPG conjugates will be characterized in one or more physiologically acceptable formulation buffers at 4° C., 20° C., and 37° C. and in rat serum at 37° C. Aliquots will be collected at the following time points: 0 min, 20 min, 40 min, 1 hr, 2 hr, 4 hr, 8 hr, 12 hr, and 24 hr. Immediately after each aliquot is collected its pH will be adjusted to 6.5 (if needed) and it will be chilled to −20° C. After all samples have been collected, they will be compared by SDS-PAGE and Western blotting.

Pharmacokinetic Studies

Pharmacokinetic studies will be conducted using PEGylated interferon beta and non-PEGylated interferon beta. Female Lewis rats (˜250 g in weight) will be used. Groups of 3 rats each will be treated as follows: (1) IV injection, non-PEGylated interferon beta, (2) SC injection, non-PEGylated interferon beta, (3) IV injection, PEGylated interferon beta, and (4) SC injection, PEGylated interferon beta. In each case, a dose of 10⁷ activity units will be used. Blood samples of 1.0 mL will be collected at the following elapsed post-injection times: 0 min, 15 min, 30 min, 1 hr, 2 hr, 4 hr, 8 hr, 24 hr. The blood will be treated with heparin and centrifuged to collect the plasma. Concentrations of interferon beta in the harvested plasma will be determined at each timepoint using the antiviral assay described above.

Immunogenicity Studies

Mouse model studies will be used to compare the immunogenicity of native interferon beta and PEGylated interferon beta. Female BALB/C mice will be used. Groups of five mice each will be treated with either native or PEGylated interferon beta. Doses of 0.5 μg interferon beta will be administered by subcutaneous injection to the neck three times each week for five weeks. Once each week, a blood sample will be obtained and plasma will be isolated for antibody detection. Total binding antibodies will be detected using plate-based ELISA assays, and neutralizing antibodies will be determined by ability to inhibit interferon beta activity in the antiviral assay described above.

Hypersensitivity Response Studies

Guinea pig model studies will be used to compare delayed-type hypersensitivity reactions resulting from treatment of native interferon beta and PEGylated interferon beta. Hartley-Durkin guinea pigs will be used. Groups of five guinea pigs each will be treated with sterile saline solution, native interferon beta, or PEGylated interferon beta. Doses of 1.0 μg interferon beta diluted to 5%, 1%, and 0.5% weight/volume with a total volume of 0.1 mL; half of each dose will be mixed with Complete Freund's Adjuvant (CFA). The first set of injections will be administered on day 1 to the flank of the guinea pigs; the injections will be separated by at least 2.5 cm and duplicated on the opposite side of the body. The second set of injections will be performed in the same manner on day 28. The third set of injections will be performed in the same manner on day 35. On days 36 and 37, the animals will be scored for the presence and severity of injection site reactions. On day 42 the fourth set of injections will be performed as before, and the animals will be scored for the presence and severity of injection site reactions on days 43 and 44.

1. A composition comprising a prodrug agent comprising:
a) a protein that induces unwanted side effects due to undesired activity at or close to the site of administration;
b) a substantially non-immunogenic polymer;
c) a covalent labile linker between said protein and said polymer, wherein said polymer substantially interferes with the activity of said protein when said polymer is covalently linked to said protein.
said protein and said polymer are connected with one or more labile covalent bonds.

2. A composition according to claim 1 wherein said protein is capable of promoting the activation of antigen presenting cells.

3. A composition according to claim 2 wherein said protein is selected from the group consisting of endothelin, interleukin-1, interleukin-4, interleukin-8, interferon-gamma, macrophage-inflammatory protein, macrophage stimulating protien, matrix metalloprotienases, thrombopoietin, transforming growth factor-beta, tumor necrosis factor-alpha, and tumor necrosis factor-beta.

4. A composition according to claim 1 wherein said protein is capable of promoting the activation of T-cells.

5. A composition according to claim 4 wherein said protein is selected from the group consisting of interferon-gamma, interleukin-1, interleukin-2, interleukin-4, interleukin-6, interleukin-7, interleukin-9, interleukin-12, and interleukin-23.

6. A composition according to claim 1 wherein said protein is a growth factor capable of inducing cellular proliferation or differentiation.

7. A composition according to claim 6 wherein said protein selected from the group consisting of vascular endothelial growth factor, tumor growth factor-beta, insulin-like growth factor, fibroblast growth factor 2, 7, or 10, and bone morphogenic proteins 2, 4, 6, and 7.

8. A composition according to claim 1 wherein said substantially non-immunogenic polymer comprises polyethylene glycol.

9. A composition according to claim 1 further comprising a pharmaceutically acceptable carrier.

10. A method for treating a disease comprising administration of a therapeutically effective amount of the composition of claim 9.

11. A method for treating a disease according to claim 10 wherein said administration is by subcutaneous injection, oral administration, or inhalation.


SEQUENCE LISTING (4 sequences)

SEQ ID NO: 1 - protein, 163 aa, Homo sapiens
SPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPS

SEQ ID NO: 2 - protein, 104 aa, Homo sapiens
QACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 3 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 4 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM